(19) United States
(12) Patent Application Publication
(10) Pub. No.: US 2019/0274641 A1
(43) Pub. Date: Sep. 12, 2019

(54) METHOD OF RADIATION DOSE REDUCTION VIA FRACTIONAL COMPUTERIZED TOMOGRAPHIC SCANNING AND SYSTEM THEREOF

(71) Applicant: Yissum Research Development Company of the Hebrew University of Jerusalem Ltd, Jerusalem (IL)

(72) Inventors: Leo JOSKOWICZ, Jerusalem (IL); Naomi SHAMUL, Rehovot (IL)

(21) Appl. No.: 15/914,127

(22) Filed: Mar. 7, 2018

(51) Int. Cl. A61B 6/03; G06T 11/00

(52) U.S. Cl. CPC A61B 6/032; G06T 7/0012; G06T 11/003

(57) ABSTRACT

There is provided a method of computer tomography (CT) volume reconstruction based on baseline sinograms, the method comprising: obtaining partial scan sinograms in a number of directions smaller than the number of directions in the baseline scan; aligning baseline sinograms and partial scan sinograms utilizing rigid registration in three-dimensional Radon space, generating configuration data informative of rays to be cast in a further repeat scan in an un-scanned direction, wherein the generating configuration data comprises: identifying, from a partial scan sinogram, rays with intensities that significantly differ from intensities of corresponding rays in an aligned baseline sinogram, identifying regions of the scanned object in which identified changed rays intersect, determining scan angles and associated rays to be cast in a further repeat scan according to the identified regions; obtaining sinograms of a repeat scan performed according to generated configuration data and processing the sinograms into an image of the object.

### TECHNICAL FIELD

The presently disclosed subject matter relates to computerized tomographic imaging and, more particularly, to interventional CT procedures.

### BACKGROUND

Computed Tomography (CT) is nowadays widely available and pervasive in routine clinical practice. Computed tomography (CT) imaging produces a 3D map of the scanned object, where the different materials are distinguished by their X-ray attenuation properties. In medicine, such a map has a great diagnostic value, making the CT scan one of the most frequent non-invasive exploration procedures practiced in almost every hospital. The number of CT scans acquired worldwide is now in the tens of millions per year and is growing at a fast pace.

A CT image is produced by exposing the patient to many X-rays with energy that is sufficient to penetrate the anatomic structures of the body. The attenuation of biological tissues is measured by comparing the intensity of the X-rays entering and leaving the body. It is now believed that ionizing radiation above a certain threshold may be harmful to the patient. The reduction of radiation dose of CT scans is nowadays an important clinical and technical issue. In CT imaging, a basic trade-off is between radiation dose and image quality.

A wide variety of approaches for CT scanning dose reduction have been proposed that attempt to overcome this issue. These can be divided into two categories. The first includes standalone methods that assume that the CT scan is independent of previous scans of the same patient. Methods in the second category use information from previous scans to compensate for noisy or incomplete data that is characteristic of low dose scans.

The vast majority of methods are in the first category. Standalone methods can be further sub-divided into several categories. First are hardware-based techniques, which include high-sensitivity sensors, focused X-ray beams, and aperture beam masking. Scanning protocol methods include sequential and “stop and shoot” scanning, automatic exposure control, and patient-specific tube current modulation. Software methods include adaptive statistical iterative image reconstruction and compressed sensing approaches that minimize the total variation of the scan under constraints of image consistency. These techniques decrease radiation exposure with reduced but clinically acceptable image quality.

Another approach for reducing the radiation dose in a single scan is selective acquisition of a fraction of scan angles, referred to as fractional, sparse-view, or few-view CT scanning. In this approach, the patient is exposed to a fraction of the radiation that is required for a full scan by reducing the number of scan angles. However, since part of the projection data is missing, severe streaking artifacts appear in the image when using standard reconstruction techniques. Different methods have been developed to reduce and/or compensate for imaging artifacts to produce clinically acceptable results. The feasibility of this approach, which is not yet currently available in commercial CT scanners, has also been investigated. Fractional scanning could be achieved, for instance, by alternating the voltage between 80-100 kV and 30-40 kV at different scan angles, thus substantially reducing the absorbed radiation at unnecessary angles.

Taking this approach a step further, Barret et al. (“Adaptive SPECT,” IEEE Trans. Med. Imag., vol. 27, no. 6, pp. 775-788, June 2008.) introduce the concept of adaptive data acquisition in CT, in which the scanner's geometric configuration and scan protocol are adjusted online to best suit the object being scanned and thus minimize the radiation dose. In another paper, Barret et al (“Instrumentation Design for Adaptive SPECT/CT.” In IEEE Nuclear Science Symposium Conference Record, 2008. NSS'08. pp. 5585-5587, 2008) a micro-CT system is presented that uses a beam-masking aperture attached to the X-ray source to shape the emitted beam and scan only a region of interest. Similarly, Chityala et al “Region of interest (ROI) computed tomography (CT): Comparison with full field of view (FFOV) and truncated CT for a human head phantom.” Proc. Of SPIE—the Int. Society for Opt. Eng., vol. 5745, no. 1, pp. 583-590, 2011) use a beam filter to obtain high image quality only within a region of interest (ROI).

A prominent technique is prior image constrained compressed sensing (PICCS), in which the optimization objective function integrates information from a previous scan. Another such method is PIRPLE, a model-based approach that integrates both a noise model and a prior image. This method performs a joint optimization procedure to simultaneously reconstruct the image and register it to the previous scan. Ma et al (“Lowdose computed tomography image restoration using previous normal-dose scan.” Medical Physics, vol. 38, no. 10, pp. 5713-5731, October 2011) bypass the need for precise registration and exploit the redundancy between the low dose repeat scan and a full dose baseline scan using nonlocal means. They report that a rough registration is sufficient to utilize the redundant information and suppress the noise-induced artifacts in the low dose reconstruction. Lee et al (“Improved compressed sensing-based cone-beam CT reconstruction using adaptive prior image constraints.” Physics in Medicine and Biology, vol. 57, no. 8, pp. 2287-307, March 2012) use the prior image as an initial starting guess for the optimization. Their method also detects possible mismatched regions and assigns them greater weight values, causing them to be updated more by the new projection data during the iterative reconstruction process. Pourmorteza et al (“Reconstruction of difference using prior images and a penalized likelihood framework.” In Proc. Int. Meeting on Fully Three-Dimensional Image Reconstruction in Radiology and Nuclear Medicine, pp. 252-5, 2015) integrate the prior image information into the consistency term of the optimization objective function to reconstruct only the difference image. The difference image can then be combined with information from the baseline scan to compute the current anatomy. This method assumes that the two scans are already registered.

Related methods perform a low dose pre-scan which serves as a prior scanning, rather than using an existing patient scan. Barret et al (“Adaptive CT for high resolution, controlled-dose, region-of-interest imaging.” Proc. IEEE Nuclear Science Symposium (NSS/MIC), pp 4154-4157, October 2009) describe a method in which a sparsely sampled scout scan is used to manually determine a region of interest which is then scanned at diagnostic quality. Barkan et al “Super-sparsely view-sampled cone-beam CT by incorporating prior data.” Journal of X-ray science and technology, vol. 21, no. 1, pp. 71-83, January 2013) use Ridgelet analysis on intermediate, low dose reconstructions to compute iterative, selective acquisition steps which enable limiting the dose level to the minimum required for sufficient image quality.

The references cited above teach background information that may be applicable to the presently disclosed subject matter. Therefore the full contents of these publications are incorporated by reference herein where appropriate for appropriate teachings of additional or alternative details, features and/or technical background.

### General Description

In CT imaging, a basic trade-off is between radiation dose and image quality. Lower doses produce imaging artifacts and increased noise, thereby reducing the image quality and limiting clinical usefulness. Since CT imaging exposes the patient to substantial X-ray ionizing radiation, radiation dose reduction is beneficial.

The present subject matter describes a new method for selection of rays and scan angles to be used in a followup scan. The followup scans can then be composed with the baseline scan to generate an image.

Advantages of this method (compared to existing methods) include: 1) diagnostic-quality image reconstruction while reducing the radiation dose 2) optimization of the radiation dose for the specific patient being scanned 3) performing optimizations in Radon space, which can be less susceptible to noise and artifacts than image space 4) lower computation times 5) fully automatic operation.

According to one aspect of the presently disclosed subject matter there is provided a method of computer tomography (CT) volume reconstruction based on a plurality of baseline sinograms obtained from a prior scanning of an object in B directions, the method comprising:
obtaining a first plurality of partial scan sinograms of an initial repeat scanning of the object in b directions out of B directions, b being substantially less than B;
aligning baseline sinograms and sinograms of the first plurality of partial scan sinograms, wherein aligning is provided by rigid registration in three-dimensional (3D) Radon space, resulting in aligned baseline sinograms;
for at least one slice, generating configuration data informative, at least, of rays to be cast in a further repeat scan in an un-scanned direction, wherein the generating configuration data comprises:
   identifying, from a sinogram of the first plurality of partial scan sinograms of the at least one slice, rays with intensities that significantly differ from intensities of corresponding rays in an aligned baseline sinogram, resulting in identified changed rays;
   identifying regions of the scanned object in which identified changed rays intersect, resulting in one or more identified regions of intersection;
   determining scan angles and associated rays to be cast in a further repeat scan according to at least part of the identified regions of intersection;
obtaining a second plurality of partial scan sinograms of a repeat scan performed in accordance with the generated configuration data;
composing baseline sinograms, at least one sinogram of the first plurality of partial scan sinograms, and at least one sinogram of the second plurality of partial scan sinograms into a composed plurality of sinograms; and
processing the composed plurality of sinograms into an image of the scanned object.

In addition to the above features, the method according to this aspect of the presently disclosed subject matter can comprise one or more of features (i) to (xv) listed below, in any desired combination or permutation which is technically possible:
i. obtaining the identified changed rays comprises computing a difference in detected intensity between a first ray of a partial scan sinogram and the corresponding ray in a baseline sinogram, and selecting the first ray if the difference exceeds a noise threshold.
ii. the noise threshold is determined according to the detected intensity of the first ray.
iii. obtaining the identified changed rays comprises computing a difference in detected intensity between a first ray of a partial scan sinogram and the corresponding ray in a baseline sinogram, and selecting the first ray if the difference exceeds a registration error threshold.
iv. the registration error threshold is determined according to the difference between the gradient of the aligned baseline sinogram for the ray's scan angle and the gradient of the partial scan sinogram for the corresponding ray's scan angle.
v. the registration error threshold is determined according to the difference between the interslice gradient of the aligned baseline sinogram for the ray's scan angle and the interslice gradient of the partial scan sinogram for the corresponding ray's scan angle.
vi. further comprising, subsequent to identifying regions of the scanned object in which identified changed rays intersect:
   assessing identified regions of intersection for likelihood of change;
   selecting one or more regions of the scanned object for rescanning according to likelihoods of change of the identified regions;
and the determining scan angles and associated rays to be cast is provided according to the selected regions.
vii. assessing identified regions of intersection for likelihood of change comprises:
   backprojecting identified rays onto the baseline scan, resulting in a likelihood map image wherein the intensity of the pixels of a region of the likelihood map image corresponds to the number of changed rays intersecting a corresponding region of the scanned object.
viii. selecting the one or more regions comprises at least one of:
   a) identifying, in the likelihood map image, pixels with intensities exceeding a threshold intensity and selecting one or more regions corresponding to the identified pixels;
   b) identifying edge pixels in the likelihood map image according to an edge threshold, and selecting one or more regions corresponding to the identified edge pixels;
   c) for pixels in the likelihood map image with an intensity higher than a local maxima threshold, setting the intensity to equal the local maxima threshold, and identifying local maxima pixels and selecting one or more regions corresponding to the identified pixels.
ix. selecting one or more regions further comprises:
   d) identifying pixels located in bands that pass through previously selected pixels and setting the intensity of the identified pixels to zero; and
   e) reducing at least one of: the threshold intensity, the edge threshold, and the local maxima threshold; and
   f) repeating at least one of steps a)-c) utilizing the respective reduced thresholds.
x. selecting one or more regions further comprises:
   g) identifying pixels located in bands that pass through previously selected pixels and setting the intensity of the identified pixels to zero; and
   h) reducing at least one of: the threshold intensity, the edge threshold, and the local maxima threshold; and
   i) repeating at least one of steps a)-c) utilizing the respective reduced thresholds.
xi. further comprising, subsequent to the selecting one or more regions:
   identifying regions that are adjacent to changed regions of adjacent slices and selecting one or more of the regions.
xii. the selecting edge pixels comprises Canny edge detection.
xiii. the selecting local maxima pixels comprises gradient descent.
xiv. the assessing identified regions of intersection for likelihoods of change comprises:
   counting the number of changed rays intersecting each identified region.
xv. the selecting regions of the scanned object for rescanning according to likelihoods of change of assessed regions comprises:
   selecting regions which are intersected by a number of changed rays which exceeds an intersecting ray threshold.

According to one aspect of the presently disclosed subject matter there is provided a computer-based volume reconstruction unit configured to operate in conjunction with a CT scanner and to provide volume reconstruction based on a plurality of baseline sinograms obtained from a prior scanning of an object in B directions, the unit comprising a processing circuitry configured:
to obtain a first plurality of partial scan sinograms of an initial repeat scanning of the object in b directions out of B directions, b being substantially less than B;
to align baseline sinograms and partial scan sinograms of the first plurality of partial scan sinograms, wherein aligning is provided by rigid registration in three-dimensional (3D) Radon space, resulting in aligned baseline sinograms;
for at least one slice, to generate configuration data informative, at least, of rays to be cast in a further repeat scan in an un-scanned direction, wherein generating configuration data comprises:
   identifying, from a sinogram of the first plurality of partial scan sinograms of the at least one slice, rays with intensities that significantly differ from intensities of corresponding rays in an aligned baseline sinogram, resulting in identified changed rays;
   identifying regions of the scanned object in which identified changed rays intersect, resulting in one or more identified regions of intersection;
   determining scan angles and associated rays to be cast in a further repeat scan according to at least part of the identified regions of intersection;
to obtain a second plurality of partial scan sinograms of a repeat scan performed in accordance with the generated configuration data;
to compose baseline sinograms, at least one sinogram of the first plurality of partial scan sinograms, and at least one sinogram of the second plurality of partial scan sinograms into a composed plurality of sinograms; and
to process the composed plurality of sinograms into an image of the scanned object.

According to one aspect of the presently disclosed subject matter there is provided a computer program product comprising computer readable storage medium program instructions, these program instructions, when read by a processor, cause the processor to perform a method of computer tomography (CT) volume reconstruction based on a plurality of baseline sinograms obtained from a prior scanning of an object in B directions, the method comprising:
obtaining a first plurality of partial scan sinograms of an initial repeat scanning of the object in b directions out of B directions, b being substantially less than B;
aligning baseline sinograms and sinograms of the first plurality of partial scan sinograms, wherein aligning is provided by rigid registration in three-dimensional (3D) Radon space, resulting in aligned baseline sinograms;
for at least one slice, generating configuration data informative, at least, of rays to be cast in a further repeat scan in an un-scanned direction, wherein the generating configuration data comprises:
   identifying, from a sinogram of the first plurality of partial scan sinograms of the at least one slice, rays with intensities that significantly differ from intensities of corresponding rays in an aligned baseline sinogram, resulting in identified changed rays;
   identifying regions of the scanned object in which identified changed rays intersect, resulting in one or more identified regions of intersection;
   determining scan angles and associated rays to be cast in a further repeat scan according to at least part of the identified regions of intersection;
obtaining a second plurality of partial scan sinograms of a repeat scan performed in accordance with the generated configuration data;
composing baseline sinograms, at least one sinogram of the first plurality of partial scan sinograms, and at least one sinogram of the second plurality of partial scan sinograms into a composed plurality of sinograms; and
processing the composed plurality of sinograms into an image of the scanned object.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to understand the invention and to see how it can be carried out in practice, embodiments will be described, by way of non-limiting examples, with reference to the accompanying drawings, in which:

FIG. 1 illustrates a functional block diagram of a CT scanning system in accordance with certain embodiments of the presently disclosed subject matter;

FIG. 2 illustrates a generalized flow diagram of volume reconstruction using selective repeat scanning in accordance with certain embodiments of the presently disclosed subject matter;

FIG. 3 illustrates a generalized flow diagram for identifying rays that have passed through a changed area of the scanned object, in accordance with certain embodiments of the presently disclosed subject matter;

FIG. 4 illustrates a generalized flow diagram for assessing the likelihood of change in regions of intersection and selecting regions with greater likelihood of change, in accordance with certain embodiments of the presently disclosed subject matter;

FIG. 5 illustrates an example of a changed region detection procedure, according to certain embodiments of the presently disclosed subject matter;

FIG. 6 illustrates steps of a method on a sample simulated input, according to certain embodiments of the presently disclosed subject matter;

FIG. 7 illustrates changed ray detection and generation of a likelihood map, according to certain embodiments of the presently disclosed subject matter; and

FIG. 8 illustrates exemplary construction results, according to certain embodiments of the presently disclosed subject matter.

### DETAILED DESCRIPTION

In the following detailed description, numerous specific details are set forth in order to provide a thorough understanding of the invention. However, it will be understood by those skilled in the art that the presently disclosed subject matter may be practiced without these specific details. In other instances, well-known methods, procedures, components and circuits have not been described in detail so as not to obscure the presently disclosed subject matter.

Unless specifically stated otherwise, as apparent from the following discussions, it is appreciated that throughout the specification discussions utilizing terms such as “processing”, “computing”, “representing”, “comparing”, “generating”, “matching”, or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities including, by way of non-limiting example, the Volume Reconstruction Unit disclosed in the present application.

The terms “non-transitory memory” and “non-transitory storage medium” used herein should be expansively construed to cover any volatile or non-volatile computer memory suitable to the presently disclosed subject matter.

The operations in accordance with the teachings herein may be performed by a computer specially constructed for the desired purposes or by a general-purpose computer specially configured for the desired purpose by a computer program stored in a non-transitory computer-readable storage medium.

Embodiments of the presently disclosed subject matter are not described with reference to any particular programming language. It will be appreciated that a variety of programming languages may be used to implement the teachings of the presently disclosed subject matter as described herein.

For purpose of illustration only, the following description is provided for parallel-beam scanning. Those skilled in the art will readily appreciate that the teachings of the presently disclosed subject matter are, likewise, applicable to fan-beam and cone-beam CT scanning.

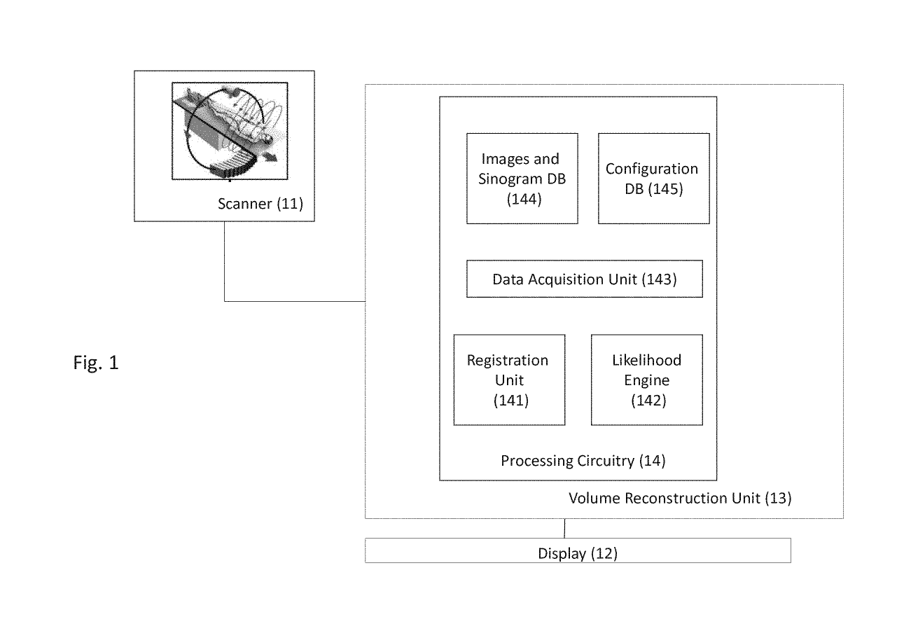

Attention is now drawn to FIG. 1 illustrating a functional diagram of a CT repeat scanning system in accordance with certain embodiments of the present subject matter.

The illustrated CT scanning system comprises a CT scanner (11) which is configured to provide selective repeat scanning and is operatively coupled to a volume reconstruction unit (13).

The volume reconstruction unit (13) comprises a processing circuitry (14) comprising a processor and a memory (not shown separately within the processing circuitry).

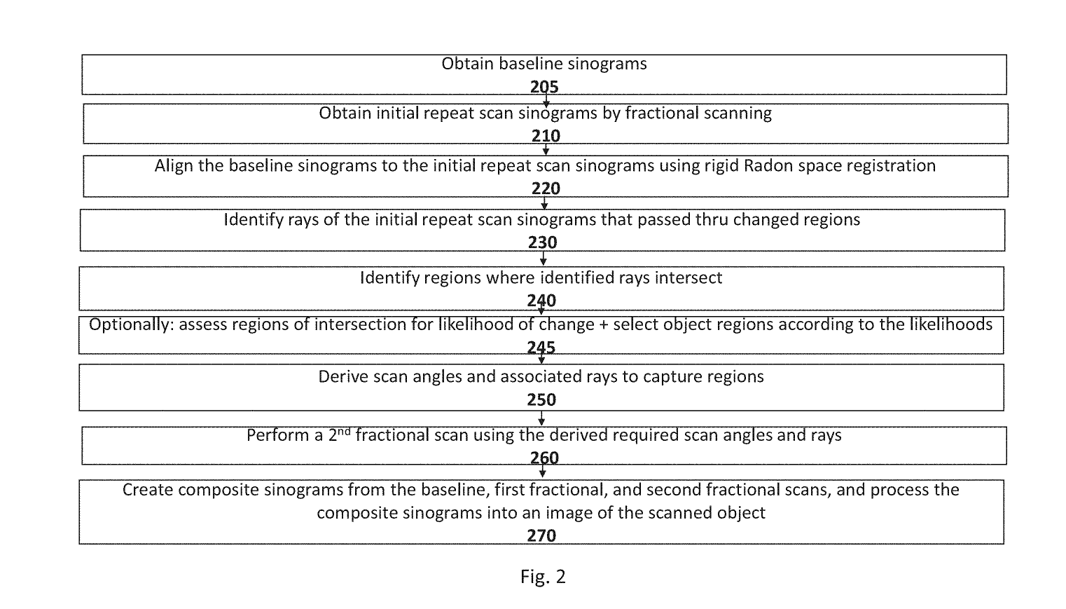

As will be further detailed below with reference to FIGS. 2-5, the processing circuitry (14) can be configured to execute several functional modules in accordance with computer-readable instructions implemented on a non-transitory computer-readable storage medium. Such functional modules are referred to hereinafter as comprised in the processing circuitry.

The processing circuitry (14) can comprise a data acquisition unit (143) configured to acquire data indicative of 3D projective measurements by the scanner (11) and to generate corresponding sinograms. Optionally, the data acquisition unit (143) can receive sinograms from the CT scanner (11). The generated sinograms (e.g. full sinograms from a baseline scan, partial sinograms from repeat scans, etc.) can be stored in an image and sinogram database (144). The database (144) can further accommodate baseline and repeat CT scans. The processing circuitry (14) can be further configured to accommodate a configuration database (145) storing data informative of, for example, scan parameters and reconstruction models usable during the volume reconstruction.

The processing circuitry (14) can comprise a registration unit (141) configured to provide registration of the baseline scan to the patient by aligning the full sinograms from a baseline scan to partial sinograms obtained by fractional repeat scanning. The registration unit (141) can be configured to perform, for example, a Radon-space rigid registration method (as described in: G. Medan, N. Shamul, L. Joskowicz. “Sparse 3D Radon space rigid registration of CT scans: method and validation study”. IEEE Transactions on Medical Imaging, February 2017) which computes the rigid registration transformation between a baseline scan and a fractional repeat scan. The Radon-space rigid registration method works by matching one-dimensional projections obtained via summation along parallel planes of both the baseline scan and the repeat scan, in a 3D extension of the 2D Radon transform. The calculation is performed entirely in projection space, and the matching is done based on maximization of normalized cross correlation. The matched projections are then used in order to construct a set of equations, the solution of which gives the parameters of the rigid registration between the scans.

The processing circuitry (14) can further comprise a likelihood engine (142) configured to provide probabilistic estimation of the likelihood of change of each voxel in a repeat scan, thereby enabling identification of regions of interest (Roe where changes are likely to have occurred. The likelihood engine (142) can be further configured to generate data informative of parameters (for example: angles and ray selections) for further selective fractional repeat scans needed to acquire additional data on certain voxels; and the CT scanner (11) can be configured to receive the generated parameter data (and/or derivatives thereof) from the volume reconstruction unit (13), and to provide selective fractional scanning accordingly.

The processing circuitry (14) can be further configured to compose the baseline and the partial sinograms into a resulting sinogram and to process the resulting sinogram to obtain a repeat scan image. The resulting repeat scan image can be transferred for rendering at a display (12) coupled to the volume reconstruction unit.

It is noted that the teachings of the presently disclosed subject matter are not bound by the specific CT scanning system described with reference to FIG. 1. Equivalent and/or modified functionality can be consolidated or divided in another manner and can be implemented in any appropriate combination of software, firmware and hardware. The volume reconstruction unit can be implemented as a suitably programmed computer.

Attention is now directed to FIG. 2, which illustrates a generalized flow chart of volume reconstruction using selective repeat CT scanning, according to some embodiments of the current subject matter.

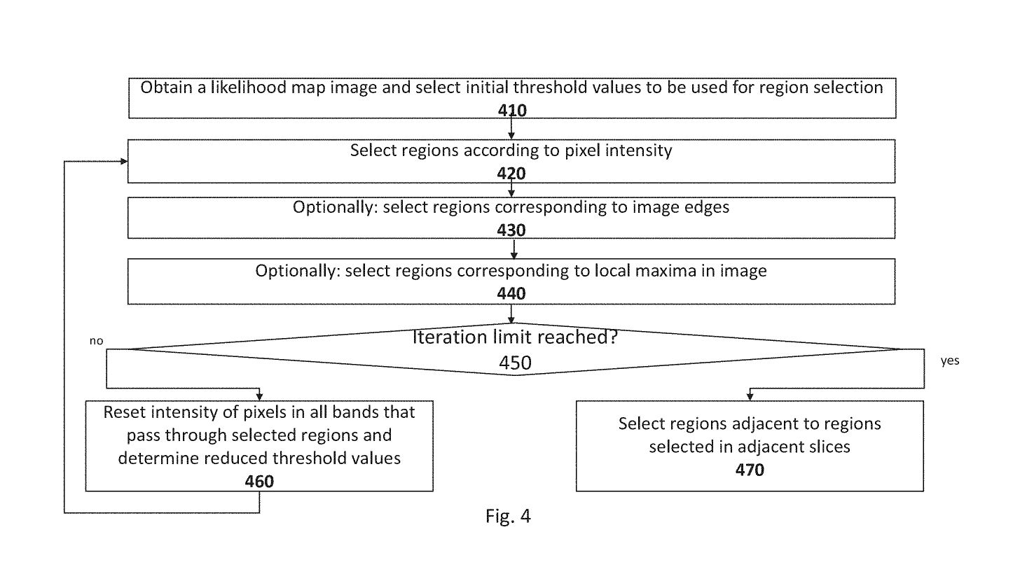

Baseline sinograms are obtained by scanning a rays (where a denotes the number of rays in a slice) for each of B directions used, in each of c slices utilized in a baseline scan (full-dose scan). In accordance with certain embodiments of the presently disclosed subject matter—at a time subsequent to obtaining (205) the baseline sinograms—the repeat scanning starts with an initial fractional repeat scanning including b directions among the B directions used for the baseline scan, and obtaining (210) initial repeat scan sinograms. The value of b and the spatial distribution of the b directions should be selected, for example, to enable acquiring sufficient information for aligning the baseline sinograms to the initial repeat scan sinograms and, thereby, registering the baseline scan to the patient, as well as for estimation of voxel change likelihoods as will be described below with reference to, for example, FIG. 4.

By way of non-limiting example, the fractional scanning can consist of scanning all a rays from a subset of b equally spaced directions out of a total of N directions for a baseline scan in a significant part of the c slices (optionally, in each of c slices). For example, b=5-20 directions out of B=180°/(angular scan resolution) that are typically used in a baseline scan. Predefined values characterizing the fractional scanning can be stored in the configuration database (145). Optionally, the number b can be selected in accordance with expected changes between the baseline scan and the repeat scan, with b increasing for higher expected changes. The initial repeat scan sinograms are hereforward denoted as SG(⋅, θ, k) where θ denotes a scan angle, and k denotes a particular slice.

After obtaining the initial repeat scan sinograms, the volume reconstruction unit can align (220) the baseline sinograms to the initial repeat scan sinograms, thereby providing rigid registration of the baseline sinograms to the patient. The registration of the baseline sinograms can be provided by the registration unit (141) via any appropriate method of registration of sinograms in 3D Radon space, some of such methods being known in the art. In accordance with certain embodiments of the presently disclosed subject matter, the registration can be provided by the method detailed in Medan etc. op. cit. The resulting aligned baseline sinograms are hereforward denoted as SF_{aligned}(⋅,θ,k).

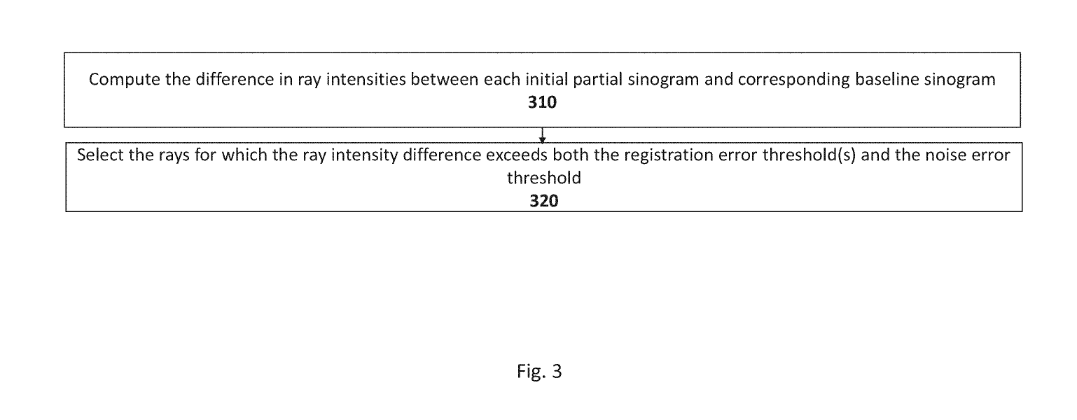

The volume reconstruction unit (e.g. the likelihood engine 142) can next identify (230) which rays in the initial repeat scan sinograms SG(⋅, θ, k) differ from the corresponding rays in the aligned baseline sinograms SF_{aligned}(⋅, θ, k) rays significantly—i.e. to an extent that indicates that the initial repeat scan sinogram ray passed through a changed region of the scanned object. To evaluate whether a pair of rays differ significantly, the volume reconstruction unit (e.g. the likelihood engine 142) can, for example, utilize a noise error threshold, one or more registration error thresholds, or combinations thereof so as to avoid identifying rays whose detected intensity has changed due to noise or due to registration error. An exemplary procedure for identifying changed rays using a noise error threshold and registration error threshold is described below, with reference to FIG. 3.

The volume reconstruction unit (13) (e.g. the likelihood engine 142) can next identify (240) regions where the identified rays intersect. The regions of the scanned object corresponding to these intersections (for example, the regions of the scanned object that the intersecting rays passed through) may contain changes in vis-a-vis the baseline scan. A region that is constrained by the intersection of multiple segments of rays from several different angles can be regarded as more likely to have changed.

The volume reconstruction unit (13) (e.g. the likelihood engine 142) can represent the regions of intersection as, for example, a set of voxel identifiers (identified by slice and x and y coordinates) of the not-yet-constructed repeat scan image.

Alternatively, the volume reconstruction unit (13) (e.g. the likelihood engine 142) can represent the regions of intersection as, for example, a slice-specific two-dimensional matrix (indexed by slice and x and y coordinates) Regionsₖ where Regionsₖ[x,y] is indicative of the number of identified rays that intersect the pixel at coordinate x,y of slice k.

Alternatively, the volume reconstruction unit (13) (e.g. the likelihood engine 142) can represent the regions of intersection as, for example, a digital image Imageₖ in which pixel intensity at Imageₖ[x,y] is indicative of the number of rays of identified rays that intersect the pixel at coordinate x,y of slice k.

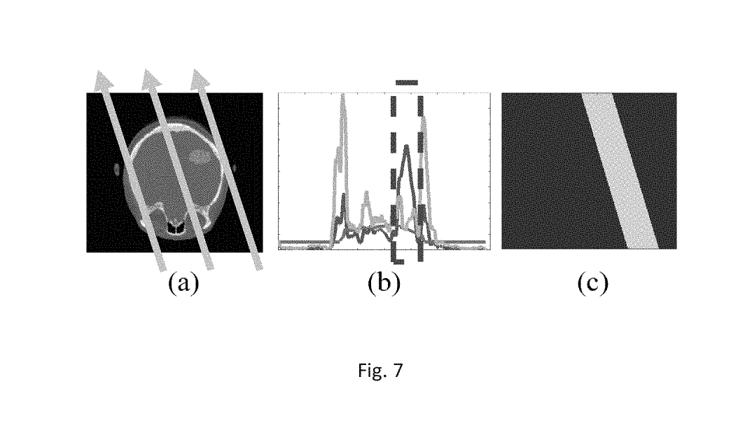

In some embodiments of the present subject matter, the volume reconstruction unit (e.g. the likelihood engine 142) identifies the regions of intersection by calculating a back-projection of each segment of selected rays—so as to produce a band whose width equals the segment's width multiplied by the ray detectors' spacing. For scan angle θ, such a band is at angle (90−θ)° with respect to the slice's horizontal axis (as shown below with reference to diagram (c) in FIG. 7). In some of these embodiments, the volume reconstruction unit (e.g. the likelihood engine 142) can then generate an image by, for example, summing the back-projections for each angle—so that intensity of pixels in a region of the image corresponds to the number of scan angles for which the corresponding pixel was identified as potentially changed. Such an image is hereforward termed a likelihood map image and is hereforward denoted LMₖ.

The volume reconstruction unit (e.g. the likelihood engine 142) can next optionally assess the regions of intersection for their likelihood of change and can select (245) for rescanning, for example, regions of the scanned object that are highly likely (as indicated by certain thresholds as described below) to have changed since the baseline scan. The volume reconstruction unit (e.g. the data acquisition unit 143) can update a maintained record of regions to indicate which regions of the scanned object have been selected (e.g. regions corresponding to voxels in slice k of a yet-to-be reconstructed CT image can be noted in a binary changed region matrix CRₖ).

In some embodiments of the presently disclosed subject matter, the volume reconstruction unit (e.g. the likelihood engine 142) assesses the regions of intersection for their likelihood of change by evaluating the number of ray segments intersecting a region.

In some embodiments of the presently disclosed subject matter, the volume reconstruction unit (e.g. the likelihood engine 142) selects a region for rescanning if the number of ray segments intersecting a region meets a particular threshold (hereforward referred to as an intersecting ray threshold). The intersecting ray threshold can be, for example, a predefined value (not greater than the total number of scan angles in the initial repeat scan) for which it is likely that this number of intersecting changed rays was caused by changes in the patient/object. The predefined value can be chosen, for example, according to the total number of scan angles in the initial repeat scan, considerations of accuracy, considerations of radiation exposure, and combinations thereof.

In some embodiments of the presently disclosed subject matter, the volume reconstruction unit (e.g. the likelihood engine 142) assesses the regions of intersection for their likelihood of change by creating a likelihood map image LMₖ (as described above) and selects regions for rescanning according to a method based on image processing of the likelihood maps, as will be described below with reference to FIG. 4.

The volume reconstruction unit (e.g. the data acquisition unit 143) can next derive (250) scan angles and associated rays for capturing the selected regions of the scanned object (which are, for example, regions that have are likely to have changed since the baseline scan).

The volume reconstruction unit (e.g. the data acquisition unit 143) can derive the scan angles and associated rays from the selected regions using, for example, mechanisms known by persons skilled in the art. A scan mask vector indicating which new rays should be cast (and detected) at a particular angle θ is hereforward denoted as SM_{θ,k}.

The scanner (11), for example, can then perform (260) the second fractional scan of the patient from the unscanned angles according to per-slice scan mask vectors SM_{θ,k}, and the volume reconstruction unit (13) (e.g. the data acquisition unit 143) can use the data from the scanner (11) to create sinograms, resulting in second fractional scan sinograms.

The volume reconstruction unit (e.g. the data acquisition unit 143) can then create (270) composite sinograms from the sinograms of the baseline, first fractional, and second fractional scans. The volume reconstruction unit (e.g. the data acquisition unit 143) can then process the composite sinograms—together with the baseline sinograms (for angles that were not rescanned)—into a reconstructed volume using standard CT image reconstruction methods.

Attention is now drawn to FIG. 3, which illustrates a generalized flow diagram for identifying rays that have passed through a changed area of the scanned object.

The volume reconstruction unit (13) (e.g. the likelihood engine 142) can compute (310) the difference in ray intensities between each ray in initial repeat scan sinogram SG(⋅, θ, k) and the corresponding ray in the aligned baseline sinogram SF_{aligned} (⋅, θ, k). D_{θ,k} will hereforward denote a vector of the per-ray absolute values of ray intensity differences for a particular scan angle θ and slice k.

More formally: the volume reconstruction unit (13) (e.g. the likelihood engine 142) can compute D_{θ,k} for each of the initial repeat scan sinograms according to the following formula:

D_{θ,k}=|SF_{aligned}(⋅,θ,k)−SG(⋅,θ,k)|

In some embodiments of the present subject matter, the volume reconstruction unit (e.g. the likelihood engine 142) can perform smoothing on D_{θ,k} following its computation. For example, the volume reconstruction unit (e.g. the likelihood engine 142) can reduce or increase values in vector D_{θ,k} to render them in accordance with the detected intensities of adjacent rays.

The volume reconstruction unit (e.g. the likelihood engine 142) can next determine (320) the rays which can be regarded as having passed through a region that changed between the two scans.

The volume reconstruction unit (e.g. the likelihood engine 142) can accomplish this, for example, by determining the rays for which the ray intensity difference exceeds an intraslice registration error threshold (hereforward designated as thresh_{θ,k}^{align}), an interslice registration threshold (hereforward designated as between_slice_thresh_{θ,k}^{align}), a noise error threshold (hereforward designated as thresh_{θ,k}^{noise}), or combinations thereof.

M_{θ,k} will hereforward denote a vector of Boolean variables indicating whether a particular ray (as indicated by the ray index) of the sinogram for scan angle θ and slice k in the initial fractional scan can be regarded to have passed through a region that changed between the two scans.

In some embodiments of the present subject matter, the volume reconstruction unit (e.g. the likelihood engine 142) can compute M_{θ,k} according to the following formula:

M_{θ,k}[s]={1 if f D_{θ,k}[s]>thresh_{θ,k}^{noise} and D_{θ,k}[s]>thresh_{θ,k}^{align} and D_{θ,k}[s]> between_slice_thresh_{θ,k}^{align}}

The resulting mask vector M_{θ,k} can sometimes include narrow segments or non-contiguous segments of changed rays. In some embodiments of the present subject matter, the volume reconstruction unit (e.g. the likelihood engine 142) can perform postprocessing on M_{θ,k}, for example: by using binary morphological operators, removing narrow segments, combining adjacent segments, or combinations thereof.

Regarding the determination of the registration error thresholds, it is noted that—by definition—for a given direction, the greater the gradient of the scan, the greater the difference caused by a slight misalignment of the scan in that direction. Consequently, it is possible to estimate the effect of misalignments between the two scans based on the approximated gradient of SG(⋅,θ,k).

In some embodiments of the present subject matter, the volume reconstruction unit (e.g. the likelihood engine 142) can compute a vector of registration thresholds corresponding to within-slice displacement for a given slice/angle, by using—for example—the following formula:

thresh_{θ,k}^{align}=|Gradient(SG(⋅,θ,k))−Gradient(SF(⋅,θ,k))|

where Gradient(.) represents the intraslice image gradient function.

In some embodiments of the present subject matter, the volume reconstruction unit (e.g. the likelihood engine 142) can compute a vector of registration thresholds corresponding to between-slice displacement for a given slice/angle, by using—for example—the following formula:

between_slice_thresh_{θ,k}^{align}=|Interslice_Gradient(SG(⋅,θ,k))−Interslice_Gradient(SF(⋅,θ,k))|

where Interslice_Gradient( ) represents the interslice gradient function.

Registration error thresholds can be computed, for example, following registration, and can be stored in the configuration database 145.

Regarding the determination of noise thresholds, it is noted that the amount of scan noise increases as the value of the sinogram scan entry increases (this is evident from simulations based on the CT noise model as reported in S. Zabic et. al, “A low dose simulation tool for CT systems with energy integrating detectors” Med. Phys. Vol 40 no. 2 Mar. 2013). The volume reconstruction unit (e.g. the likelihood engine 142) can compute per-angle noise thresholds according to, for example, the following formula:

thresh_{θ,k}^{noise}=cₙₒᵢₛₑ*SG(⋅,θ,k)

where Cₙₒᵢₛₑ is, for example, an empirically chosen constant dependent on the scan protocol (by way of non-limiting example: 0.0008).

Noise thresholds can be computed, for example, following the initial partial scan, and can be stored in the configuration database 145.

Attention is now directed to FIG. 4, which illustrates a generalized flow chart for selecting regions of the scanned object with likelihood of change—according to some embodiments of the present subject matter.

To begin, the volume reconstruction unit (e.g. the likelihood engine 142) can obtain (410) a likelihood map image LMₖ for the particular slice k (the likelihood map image is described above, with reference to FIG. 2).

The method can conduct several (for example: 3) iterations. Each iteration can utilize several different thresholds (described below), to identify pixels in the likelihood map image LMₖ with characteristics that indicate that corresponding regions of the scanned object are likely to have changed since the baseline scan. Each iteration can utilize progressively lower thresholds for the identification of pixels. The chosen thresholds can, for example, affect the tradeoff between image equality and radiation dose.

For clarity and ease of description, the description below describes utilization of a binary matrix CRₖ for indicating the regions of the scanned object that have been thus identified.

Subsequent to the completion of the iterations, exemplary binary matrix CRₖ can have, for example, a binary ‘1’ stored in the matrix entry whose coordinates match the coordinates of the pixels that correspond to regions assessed as having high likelihood of change. By way of non-limiting example, it is noted that if CRₖ [i,j] is 1, then voxel G[i,j,k] of yet-to-be-constructed image G can be regarded as likely to differ from the baseline scan.

In some embodiments of the present subject matter, the volume reconstruction unit (e.g. the likelihood engine 142) can subsequently receive, for example, binary matrix CRₖ or another representation of identified regions, and use the regions for deriving the scan angles and rays for a second repeat scan, as described above with reference to FIG. 2.

It will be clear to a person skilled in the art that the volume reconstruction unit (e.g. the likelihood engine 142) can select the regions corresponding to an identified pixel at any time after the pixel has been identified.

For the initial iteration, the volume reconstruction unit (e.g. the likelihood engine 142) can determine (410) threshold values such as, for example: threshold intensity, strong edge threshold, weak edge threshold, and local maxima threshold. These thresholds are described in detail below.

The threshold intensity should be selected to be, for example, larger or smaller in a manner proportionate to the maximal intensity in each likelihood map LMₖ.

By way of non-limiting example, the threshold intensity for the initial iteration can be calculated according to the following equation:

ThresholdIntensity=0.5*max(LMₖ)

where max( ) denotes the maximum pixel intensity in the image LMₖ.

Optionally, the volume reconstruction unit (e.g. the likelihood engine 142) can perform preprocessing on the image LMₖ by increasing the contrast (for example: by using a gamma filter). Optionally, the volume reconstruction unit (e.g. the likelihood engine 142) can perform preprocessing on the image by increasing the blur (for example by using a Gaussian filter). This preprocessing can remove star-shaped halos which can surround pixels derived from areas with many intersecting changed rays (and thus high likelihoods of change).

The volume reconstruction unit (e.g. the likelihood engine 142) can next identify (420) pixels in LMₖ based on, for example, pixel intensity. By way of non-limiting example, the volume reconstruction unit (e.g. the likelihood engine 142) can identify pixels with intensity meeting ThresholdIntensity. The volume reconstruction unit (e.g. the likelihood engine 142) can then select the scanned object regions corresponding to the identified pixels by, for example, setting CRₖ matrix entries with the same x and y coordinates as the identified pixels to ‘1’.

The volume reconstruction unit (e.g. the likelihood engine 142) can optionally additionally identify (430) edge pixels in LMₖ according to at least one edge threshold. By way of non-limiting example, the volume reconstruction unit (e.g. the likelihood engine 142) can identify pixels belonging to edges of the image using, for example, a Canny edge detector. A Canny edge detector can apply, for example, two thresholds (strong edge threshold and weak edge threshold) to the gradient of the input, to distinguish between strong edges and weak edges, and, for example, fill in gaps in the strong edges using the weak edges (cf. I. Canny “A computational approach to edge detection”, IEEE Trans Pattern Anal. Mach. Intell. Vol. PAMI-8 no. 6 pp. 679-698, November 1986). The volume reconstruction unit (e.g. the likelihood engine 142) can then select the scanned object regions corresponding to the identified pixels by, for example, setting CRₖ matrix entries with the same x and y coordinates as the identified pixels, to ‘1’.

To preprocess LMₖ for optional detection of local maxima, the volume reconstruction unit (e.g. the likelihood engine 142) can reduce the intensity values of all pixels in LMₖ that are above local maxima threshold to the value of local maxima threshold. This preprocessing can increase the area of the local maxima and can cause the local maxima to better correspond to the actual changed regions.

The volume reconstruction unit (e.g. the likelihood engine 142) can optionally additionally identify (440) local maxima pixels in LMₖ. By way of non-limiting example, the volume reconstruction unit (e.g. the likelihood engine 142) can determine which pixels are local maxima by using, for example, a gradient-descent algorithm. The volume reconstruction unit (e.g. the likelihood engine 142) can then select the scanned object regions corresponding to the identified pixels by, for example, setting CRₖ[ ] matrix entries with the same x and y coordinates as the identified pixels to ‘1’.

The volume reconstruction unit (e.g. the likelihood engine 142) can check (450) whether the required number of iterations have been executed, and, if not, it can prepare for another iteration by resetting (460) (to zero) the intensities of all pixels located in bands that pass through the selected regions. In so doing, the effects of the initially identified changed regions can be removed from LMₖ. It is recalled that in some embodiments bands are defined by a back-projection of a segment of selected rays—so that the width of the band equals the segment's width, times the ray detectors' spacing. The volume reconstruction unit (e.g. the likelihood engine 142) can determine (460) new (and reduced) values for some or all of the thresholds (e.g. ThresholdIntensity, edge threshold, local maxima threshold etc.) for the upcoming iteration (420).

In some embodiments of the present subject matter, the volume reconstruction unit (e.g. the likelihood engine 142) can, in one or more of the iterations, omit the identification of pixels with intensity meeting the ThresholdIntensity, and the selection of regions corresponding to the pixels.

Following the completion of all iterations, the volume reconstruction unit (e.g. the likelihood engine 142) can additionally identify (470) regions that are adjacent to regions that were selected in adjacent slices. The volume reconstruction unit (e.g. the likelihood engine 142) can then select one or more of the regions by, for example, setting CRₖ[ ] matrix entries with the same x and y coordinates to ‘1’.

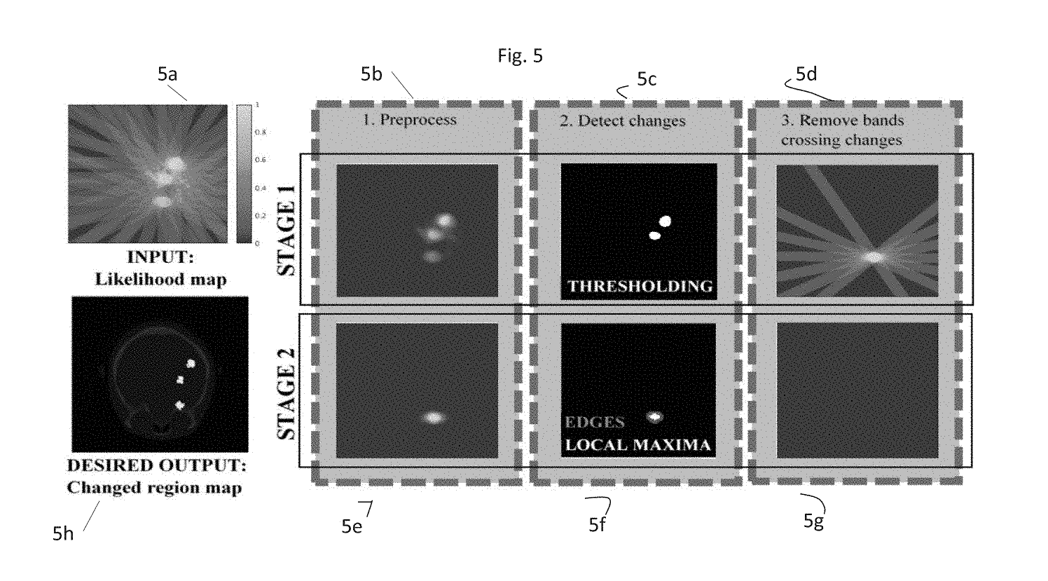

Attention is now drawn to FIG. 5, which illustrates an example of the changed region detection procedure, according to certain embodiments of the presently disclosed subject matter. FIG. 5a depicts an image representation of a likelihood map for a slice of an initial repeat scan. It shows changed regions of different intensities (likelihoods) as well as starburst “halos” surrounding the changed regions. FIG. 5b shows the removal of the “halos” by preprocessing i.e. increasing the contrast and smoothing. FIG. 5c shows initially identified changed regions (detected according to the intensity threshold). FIG. 5d shows the effects of removing bands which cross previously identified regions. FIG. 5e shows the image following removal of the bands. FIG. 5f shows the changed region identified in the second iteration using edges and local maxima. FIG. 5g shows that the remaining image is empty, so that a third iteration is not necessary. FIG. 5h shows the map of regions for rescanning.

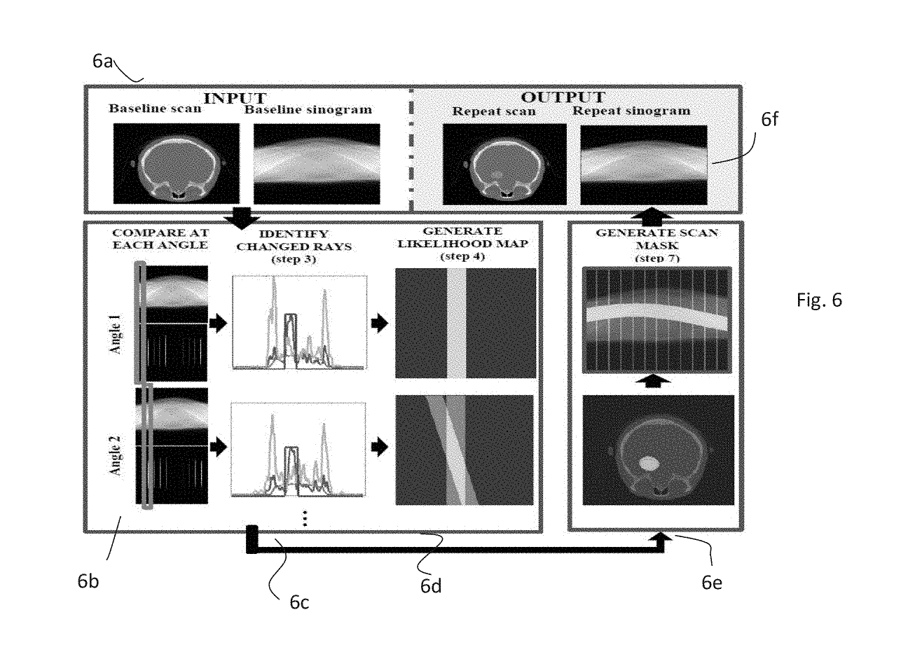

Attention is now drawn to FIG. 6, which illustrates steps of the method on a sample simulated input, according to some embodiments of the present subject matter. In FIG. 6a a sparse subset of scan angles is acquired and registered with the baseline sinogram. In FIG. 6b each scan angle is compared with the corresponding angle in the aligned baseline. In FIG. 6c changed rays are backprojected to produce a likelihood map LMₖ which indicates regions that have a high likelihood of being changed. In FIG. 6d the changed region map CRₖ is computed based on the likelihood map, indicating which pixels were changed. In FIG. 6e the scan mask SMₖ is computed based on this map. The remaining angles are scanned according to the mask, and the missing values are completed from the baseline.

Attention is now drawn to FIG. 7, which illustrates changed ray detection and generation of the likelihood map, according to some embodiments of the present subject matter. FIG. 7a shows the generation of a simulated repeat scan by adding a changed region to a patient scan. FIG. 7b shows the absolute difference between the sinograms at an angle of a particular slice in conjunction with a noise threshold (red) and a misalignment threshold (yellow)—and the indices with values larger than both thresholds are marked as changed (purple). FIG. 7c shows pixels whose backprojected rays pass through in the likelihood map.

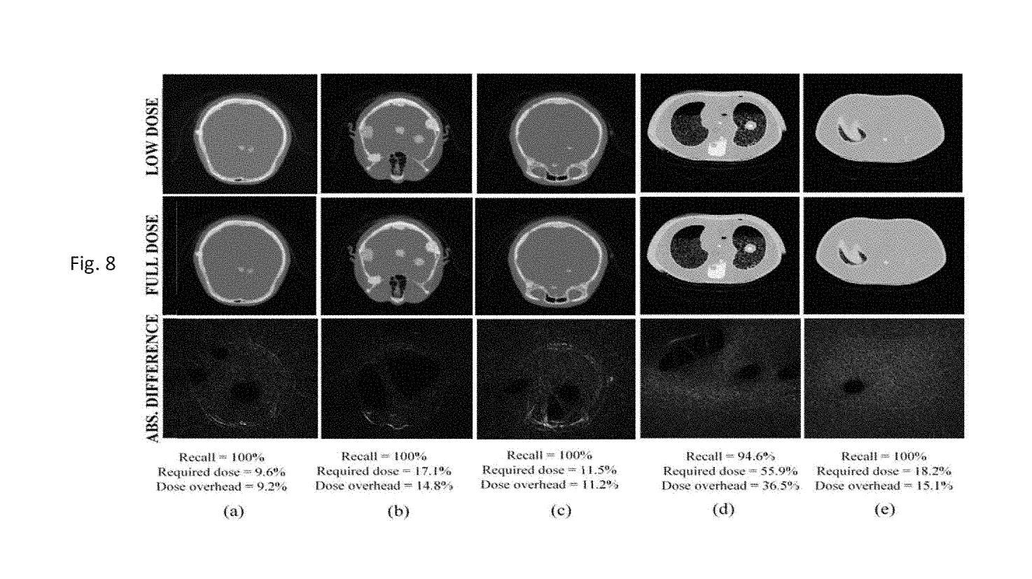

Attention is now drawn to FIG. 8, which illustrates exemplary construction results according to some embodiments of the presently disclosed subject matter. The top row illustrates reconstruction in accordance with some embodiments of the presently disclosed subject matter. The middle row illustrates reconstruction obtained from the complete repeat scan sinogram. The bottom row illustrates the absolute difference between the two images. Images a-c show slices from scans with simulated changes. Images d-e show slices from a real phantom scan.

It is to be understood that the invention is not limited in its application to the details set forth in the description contained herein or illustrated in the drawings. The invention is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

It will also be understood that the system according to the invention may be, at least partly, implemented on a suitably programmed computer. Likewise, the invention contemplates a computer program being readable by a computer for executing the method of the invention. The invention further contemplates a non-transitory computer-readable memory tangibly embodying a program of instructions executable by the computer for executing the method of the invention.

Those skilled in the art will readily appreciate that various modifications and changes can be applied to the embodiments of the invention as hereinbefore described without departing from its scope, defined in and by the appended claims.

1. A method of computer tomography (CT) volume reconstruction based on a plurality of baseline sinograms obtained from a prior scanning of an object in B directions, the method comprising:
obtaining a first plurality of partial scan sinograms of an initial repeat scanning of the object in b directions out of B directions, b being substantially less than B;
aligning baseline sinograms and sinograms of the first plurality of partial scan sinograms, wherein aligning is provided by rigid registration in three-dimensional (3D) Radon space, resulting in aligned baseline sinograms;
for at least one slice, generating configuration data informative, at least, of rays to be cast in a further repeat scan in an un-scanned direction, wherein the generating configuration data comprises:
identifying, from a sinogram of the first plurality of partial scan sinograms of the at least one slice, rays with intensities that significantly differ from intensities of corresponding rays in an aligned baseline sinogram, resulting in identified changed rays;
identifying regions of the scanned object in which identified changed rays intersect, resulting in one or more identified regions of intersection;
determining scan angles and associated rays to be cast in a further repeat scan according to at least part of the identified regions of intersection;
obtaining a second plurality of partial scan sinograms of a repeat scan performed in accordance with the generated configuration data;
composing baseline sinograms, at least one sinogram of the first plurality of partial scan sinograms, and at least one sinogram of the second plurality of partial scan sinograms into a composed plurality of sinograms; and
processing the composed plurality of sinograms into an image of the scanned object.

2. The method of claim 1, wherein obtaining the identified changed rays comprises computing a difference in detected intensity between a first ray of a partial scan sinogram and the corresponding ray in a baseline sinogram, and selecting the first ray if the difference exceeds a noise threshold.

3. The method of claim 2, wherein the noise threshold is determined according to the detected intensity of the first ray.

4. The method of claim 1, wherein obtaining the identified changed rays comprises computing a difference in detected intensity between a first ray of a partial scan sinogram and the corresponding ray in a baseline sinogram, and selecting the first ray if the difference exceeds a registration error threshold.

5. The method of claim 4, wherein the registration error threshold is determined according to the difference between the gradient of the aligned baseline sinogram for the ray's scan angle and the gradient of the partial scan sinogram for the corresponding ray's scan angle.

6. The method of claim 4, wherein the registration error threshold is determined according to the difference between the interslice gradient of the aligned baseline sinogram for the ray's scan angle and the interslice gradient of the partial scan sinogram for the corresponding ray's scan angle.

7. The method of claim 1, further comprising, subsequent to identifying regions of the scanned object in which identified changed rays intersect:
assessing identified regions of intersection for likelihood of change;
selecting one or more regions of the scanned object for rescanning according to likelihoods of change of the identified regions;
and wherein the determining scan angles and associated rays to be cast is provided according to the selected regions.

8. The method of claim 7, wherein assessing identified regions of intersection for likelihood of change comprises:
backprojecting identified rays onto the baseline scan, resulting in a likelihood map image wherein the intensity of the pixels of a region of the likelihood map image corresponds to the number of changed rays intersecting a corresponding region of the scanned object.

9. The method of claim 8, wherein selecting the one or more regions comprises at least one of:
a) identifying, in the likelihood map image, pixels with intensities exceeding a threshold intensity and selecting one or more regions corresponding to the identified pixels;
b) identifying edge pixels in the likelihood map image according to an edge threshold, and selecting one or more regions corresponding to the identified edge pixels;
c) for pixels in the likelihood map image with an intensity higher than a local maxima threshold, setting the intensity to equal the local maxima threshold, and
identifying local maxima pixels and selecting one or more regions corresponding to the identified pixels.

10. The method of claim 9, wherein the selecting one or more regions further comprises:
d) identifying pixels located in bands that pass through previously selected pixels and setting the intensity of the identified pixels to zero; and
e) reducing at least one of: the threshold intensity, the edge threshold, and the local maxima threshold; and
f) repeating at least one of steps a)-c) utilizing the respective reduced thresholds.

11. The method of claim 10, wherein the selecting one or more regions further comprises:
g) identifying pixels located in bands that pass through previously selected pixels and setting the intensity of the identified pixels to zero; and
h) reducing at least one of: the threshold intensity, the edge threshold, and the local maxima threshold; and
i) repeating at least one of steps a)-c) utilizing the respective reduced thresholds.

12. The method of claim 9, further comprising, subsequent to the selecting one or more regions:
identifying regions that are adjacent to changed regions of adjacent slices and selecting one or more of the regions.

13. The method of claim 9, wherein the selecting edge pixels comprises Canny edge detection.

14. The method of claim 9, wherein the selecting local maxima pixels comprises gradient descent.

15. The method of claim 7, wherein the assessing identified regions of intersection for likelihoods of change comprises:
counting the number of changed rays intersecting each identified region.

16. The method of claim 15, wherein the selecting regions of the scanned object for rescanning according to likelihoods of change of assessed regions comprises:
selecting regions which are intersected by a number of changed rays which exceeds an intersecting ray threshold.

17. A computer-based volume reconstruction unit configured to operate in conjunction with a CT scanner and to provide volume reconstruction based on a plurality of baseline sinograms obtained from a prior scanning of an object in B directions, the unit comprising a processing circuitry configured:
to obtain a first plurality of partial scan sinograms of an initial repeat scanning of the object in b directions out of B directions, b being substantially less than B;
to align baseline sinograms and partial scan sinograms of the first plurality of partial scan sinograms, wherein aligning is provided by rigid registration in three-dimensional (3D) Radon space, resulting in aligned baseline sinograms;
for at least one slice, to generate configuration data informative, at least, of rays to be cast in a further repeat scan in an un-scanned direction, wherein generating configuration data comprises:
identifying, from a sinogram of the first plurality of partial scan sinograms of the at least one slice, rays with intensities that significantly differ from intensities of corresponding rays in an aligned baseline sinogram, resulting in identified changed rays;
identifying regions of the scanned object in which identified changed rays intersect, resulting in one or more identified regions of intersection;
determining scan angles and associated rays to be cast in a further repeat scan according to at least part of the identified regions of intersection;
to obtain a second plurality of partial scan sinograms of a repeat scan performed in accordance with the generated configuration data;
to compose baseline sinograms, at least one sinogram of the first plurality of partial scan sinograms, and at least one sinogram of the second plurality of partial scan sinograms into a composed plurality of sinograms; and
to process the composed plurality of sinograms into an image of the scanned object.

18. A computer program product comprising a computer readable storage medium retaining program instructions, these program instructions, when read by a processor, cause the processor to perform a method of computer tomography (CT) volume reconstruction based on a plurality of baseline sinograms obtained from a prior scanning of an object in B directions, the method comprising:
obtaining a first plurality of partial scan sinograms of an initial repeat scanning of the object in b directions out of B directions, b being substantially less than B;
aligning baseline sinograms and sinograms of the first plurality of partial scan sinograms, wherein aligning is provided by rigid registration in three-dimensional (3D) Radon space, resulting in aligned baseline sinograms;
for at least one slice, generating configuration data informative, at least, of rays to be cast in a further repeat scan in an un-scanned direction, wherein the generating configuration data comprises:
identifying, from a sinogram of the first plurality of partial scan sinograms of the at least one slice, rays with intensities that significantly differ from intensities of corresponding rays in an aligned baseline sinogram, resulting in identified changed rays;
identifying regions of the scanned object in which identified changed rays intersect, resulting in one or more identified regions of intersection;
determining scan angles and associated rays to be cast in a further repeat scan according to at least part of the identified regions of intersection;
obtaining a second plurality of partial scan sinograms of a repeat scan performed in accordance with the generated configuration data;
composing baseline sinograms, at least one sinogram of the first plurality of partial scan sinograms, and at least one sinogram of the second plurality of partial scan sinograms into a composed plurality of sinograms; and
processing the composed plurality of sinograms into an image of the scanned object.
